(19) United States
(12) Patent Application Publication
(10) Pub. No.: US 2026/0263817 A1
(43) Pub. Date: Sep. 10, 2026

(54) IMPLANTABLE MEDICAL DEVICE INCLUDING SURROUND

(71) Applicant: MEDTRONIC, INC., Minneapolis, MN (US)

(72) Inventors: Rajesh V. Iyer, Eden Prairie, MN (US); Andrew Thom, Maple Grove, MN (US); Paul B. Young, New Richmond, WI (US); Teresa A. Whitman, Dayton, MN (US); Matthew D. Bonner, Plymouth, MN (US); Erik J. Hovland, Minnetonka, MN (US); Andrew J. Ries, Lino Lakes, MN (US); Raymond W. Usher, Coon Rapids, MN (US)

(21) Appl. No.: 18/881,569

(22) PCT Filed: Jun. 12, 2023
(86) PCT No.: PCT/IB2023/056052

§ 371 Date: Jan. 6, 2025

Related U.S. Application Data
(60) Provisional application No. 63/359,192, filed on Jul. 7, 2022.

(51) Int. Cl. A61N 1/375; A61N 1/362

(52) U.S. Cl. CPC A61N 1/37518; A61N 1/362; A61N 1/3756

(57) ABSTRACT

Various embodiments of an implantable medical device are disclosed. The device includes a housing having a first major surface and a second major surface, electronic components disposed within the housing, and an electrode disposed on the first major surface of the housing and electrically connected to the electronic components. The device further includes a surround having a body that includes a first major surface, a second major surface, and an opening disposed in the first major surface of the body. The surround is adapted to receive within the body at least a portion of the housing. Further, the surround also includes a fixation component disposed on or through the body of the surround and adapted to attach the surround to tissue of a patient. The electrode extends through the opening of the body of the surround and is adapted to be in contact with the tissue of the patient.

This application claims the benefit of U.S. Provisional Patent Application Ser. No. 63/359,192, filed 7 Jul. 2022, the entire content of which is incorporated herein by reference.

### TECHNICAL FIELD

This disclosure generally relates to implantable medical devices and more particularly to implantable medical devices that include a surround.

### BACKGROUND

Implantable medical devices such as an implantable pacemaker can deliver pacing pulses to a patient's heart and monitor conditions of the patient's heart. In some examples, the implantable pacemaker includes a pulse generator and one or more electrical leads. The pulse generator may, for example, be implanted in a small pocket in the patient's chest. The electrical leads can be coupled to the pulse generator, which may contain circuitry that generates pacing pulses and/or senses cardiac electrical activity. The electrical leads may extend from the pulse generator to a target site (e.g., an atrium and/or a ventricle) such that electrodes at the distal ends of the electrical leads are positioned at the target site. The pulse generator may provide electrical stimulation to the target site and/or monitor cardiac electrical activity at the target site via the electrodes.

Other implantable pacemakers are adapted to be implanted entirely within a chamber of the heart. Such pacemakers can be referred to as intracardiac pacing devices or leadless pacing devices and can include one or more electrodes on their outer housings to deliver therapeutic electrical signals and/or sense intrinsic depolarizations of the heart. Such pacemakers can be positioned within or outside of the heart and, in some examples, can be anchored to a wall of the heart (e.g., epicardium) via a fixation component. Pacemakers can also be placed on a surface of the heart and fixed to tissue using a fixation component.

### SUMMARY

The techniques of this disclosure generally relate to an implantable medical device (IMD) that includes a housing and a surround that is adapted to receive the housing. At least a portion of the IMD is disposed within the surround. The surround can include a body and one or more fixation components disposed on or through the body. The one or more fixation components can be adapted to attach the surround to tissue of a patient such that an electrode of the IMD is in contact with the tissue and can transmit or receive one or more electrical signals to or from the tissue.

This disclosure includes without limitation the following clauses:

Clause 1: An implantable medical device that includes a housing having a first major surface and a second major surface, electronic components disposed within the housing, and an electrode disposed on the first major surface of the housing and electrically connected to the electronic components. The device further includes a surround that includes a body having a first major surface, a second major surface, and an opening disposed in the first major surface of the body. The surround is adapted to receive within the body at least a portion of the housing. Further, the surround also includes a fixation component disposed on or through the body of the surround and adapted to attach the surround to tissue of a patient. The electrode extends through the opening of the body of the surround and is adapted to be in contact with the tissue of the patient.

Clause 2: The device of Clause 1, where the fixation component includes a tine.

Clause 3: The device of Clause 1, where the fixation component includes a suture hole.

Clause 4: The device of any one of Clauses 1-3, where the body of the surround includes a biocompatible material.

Clause 5: The device of Clause 4, where the biocompatible material of the body of the surround includes PEEK.

Clause 6: The device of any one of Clauses 1-5, where the opening of the body of the surround is adapted to receive the housing such that at least one side surface of the housing is disposed within the surround.

Clause 7: The device of Clause 6, where each side surface of the housing is disposed within the body of the surround.

Clause 8: The device of any one of Clauses 1-7, where the surround further includes a second opening disposed in the second major surface of the body of the surround, where a perimeter of the second opening overlaps the housing in a direction orthogonal to the second major surface of the body such that the housing is prevented from being removed from the surround through the second opening.

Clause 9: The device of any one of Clauses 1-8, where the body of the surround further includes a lip disposed on the first major surface of the body of the surround that defines the opening, where the lip is adapted to be disposed on a perimeter of the first major surface of the housing that faces through the opening such that at least a portion of the perimeter of the first major surface of the housing is covered by the lip.

Clause 10: The device of any one of Clauses 1-9, where the surround is adapted such that the housing is press-fit into the body of the surround.

Clause 11: The device of any one of Clauses 1-9, where the housing is adhered to the body of the surround.

Clause 12: The device of any one of Clauses 1-9, where the body of the surround includes a first portion and a second portion connected to the first portion.

Clause 13: The device of any one of Clauses 1-12, further including a hermetic assembly that forms a part of the housing. The hermetic assembly includes a dielectric substrate having a first major surface and a second major surface, where the first major surface of the dielectric substrate defines at least a portion of the first major surface of the housing; and a ferrule hermetically sealed to the dielectric substrate. The ferrule is connected to an edge of a second portion of the housing.

Clause 14: The device of Clause 13, where the ferrule is laser bonded to the dielectric substrate.

Clause 15: The device of any one of Clauses 13-14, where at least a portion of the ferrule is disposed within the body of the surround.

Clause 16: The device of any one of Clauses 13-15, where the dielectric substrate includes sapphire.

Clause 17: The device of any one of Clauses 13-16, where the ferrule is adapted to provide a return path for electrical energy directed to the tissue by the electrode.

Clause 18: The device of any one of Clauses 13-17, where the ferrule includes titanium.

Clause 19: The device of any one of Clauses 1-18, where the housing of the implantable medical device includes a ceramic material.

Clause 20: The device of any one of Clauses 1-19, where the electronic components include a power source and a charging coil electrically connected to the power source.

Clause 21: A surround including a body that includes a first major surface and a second major surface; an opening disposed in the first major surface of the body, where the surround is adapted to receive within the body at least a portion of a housing of an implantable medical device; and a fixation component disposed on or through the body of the surround and adapted to attach the surround to tissue of a patient.

Clause 22: The surround of Clause 21, where the fixation component includes a tine.

Clause 23: The surround of Clause 21, where the fixation component includes a suture hole.

Clause 24: The surround of any one of Clauses 21-23, where the body of the surround includes a biocompatible material.

Clause 25: The surround of Clause 24, where the biocompatible material of the surround includes PEEK.

Clause 26: The surround of any one of Clauses 21-25, where the opening of the body of the surround is adapted to receive the housing such that at least one side surface of the housing is disposed within the surround.

Clause 27: The surround of clause 26, where each side surface of the housing is disposed within the body of the surround.

Clause 28: The surround of any one of Clauses 21-27, where the surround further includes a second opening disposed in the second major surface of the body of the surround, where a perimeter of the second opening is adapted to overlap the housing in a direction orthogonal to the second major surface of the body such that the housing is prevented from being removed from the surround through the second opening.

Clause 29: The surround of any one of Clauses 21-28, where the body of the surround further includes a lip disposed on the first major surface of the surround that defines the opening, where the lip is adapted to be disposed on a perimeter of the first major surface of the housing that faces through the opening such that at least a portion of the perimeter of the first major surface of the housing is covered by the lip.

Clause 30: The surround of any one of Clauses 21-29, where the surround is adapted such that the housing can be press-fit into the body of the surround.

Clause 31: A method including disposing an opening in a first major surface of a body of a surround; disposing a fixation component on or through the body of the surround; and connecting the surround to a housing of an implantable medical device. At least a portion of the housing is disposed within the body of the surround. The housing includes a first major surface and a second major surface, where an electrode disposed on the first major surface of the housing extends through the opening of the body of the surround.

Clause 32: The method of Clause 31, where the fixation component includes a tine.

Clause 33: The method of Clause 32, where the tine extends through the body of the surround.

Clause 34: The method of Clause 31, where the fixation component includes a suture hole.

Clause 35: The method of any one of Clauses 31-34, where the body of the surround includes a first portion and a second portion connected to the first portion

Clause 36: The method of any one of Clauses 31-35, where connecting the surround to the implantable medical device includes molding the surround onto the housing of the implantable medical device.

Clause 37: The method of any one of Clauses 31-35, where connecting the surround to the implantable medical device includes press-fitting the implantable medical device into the body of the surround.

Clause 38: The method of any one of Clauses 31-35, where connecting the surround to the implantable medical device includes adhering the surround to the implantable medical device.

Clause 39: The method of any one of Clauses 31-38, further including disposing a second opening in the second major surface of the body of the surround, where a perimeter of the second opening overlaps the housing of the implantable medical device in a direction orthogonal to the second major surface of the body such that the implantable medical device is prevented from being removed from the surround through the second opening.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic view of one embodiment of an implantable medical device disposed within a body of a patient.

FIG. 2 is a schematic perspective view of the implantable medical device of FIG. 1.

FIG. 3 is a schematic top plan view of the implantable medical device of FIG. 1.

FIG. 4 is a schematic bottom plan view of the implantable medical device of FIG. 1.

FIG. 5 is a schematic cross-section view of the implantable medical device of FIG. 1.

FIG. 6 is a schematic perspective view of a fixation component of the implantable medical device of FIG. 1.

FIG. 7 is a flowchart of one embodiment of a technique for assembling the implantable medical device of FIG. 1.

FIG. 8 is a schematic perspective view of another embodiment of a surround.

FIG. 9 is a schematic perspective view of an insertion tool that can be utilized to dispose the implantable medical device of FIG. 1 within the body of the patient.

FIG. 10 is a schematic cross-section view of the insertion tool of FIG. 9.

FIG. 11 is a schematic cross-section view of a portion of the insertion tool of FIG. 9 with tines of the implantable medical device of FIG. 1 retracted within the tool.

FIG. 12 is a schematic cross-section view of the portion of the insertion tool of FIG. 11 with the tines of the implantable medical device extended beyond a body of a surround of the implantable medical device.

FIG. 13 is a schematic cross-section view of another embodiment of an implantable medical device.

### DETAILED DESCRIPTION

In general, the present disclosure provides various embodiments of an implantable medical device (IMD) that includes a housing and a surround that is adapted to receive the housing. At least a portion of the IMD is disposed within the surround. The surround can include a body and one or more fixation components disposed on or through the body. The one or more fixation components can be adapted to attach the surround to tissue of a patient such that an electrode of the IMD is in contact with the tissue and can transmit or receive one or more electrical signals to or from the tissue.

IMDs typically include a housing that includes a biocompatible material. Some IMDs include metallic housings that can provide one or more electrodes that transmit or receive electrical signals. Such metallic housings can, however, be expensive to manufacture. Recently, some IMDs include ceramic or other nonconductive housings that can be less expensive to manufacture than metallic housings. These ceramic housings can, however, be challenging to manufacture such that they include one or more fixation components that are adapted to attach the IMDs to tissue of a patient as such components add complexity to the design and can also increase manufacturing costs.

One or more embodiments of IMDs described herein can provide various advantages over currently-available devices. For example, one or more embodiments of an IMD can include a surround that is adapted to receive at least a portion of a housing of the IMD. In one or more embodiments, the IMD can be press-fit into the surround or attached to the surround using any suitable technique. The surround can include one or more fixation components disposed on or through a body of the surround. The fixation components can be connected to the surround using any suitable technique and are adapted to attach the surround to the tissue of the patient. Disposing the fixation component on or through the surround can lower the costs of manufacturing the device when compared to disposing such component on the housing. Further, the surround can extend over one or more edges of the housing of the device to prevent such edges from engaging tissue of the patient. In one or more embodiments, the surround can also include a charging coil for receiving electromagnetic energy from an external charging device and provide such energy to one or more power sources (e.g., one or more batteries) disposed within the IMD. The surround can also include a shape or contour that can better match anatomy of an organ to which the surround is attached.

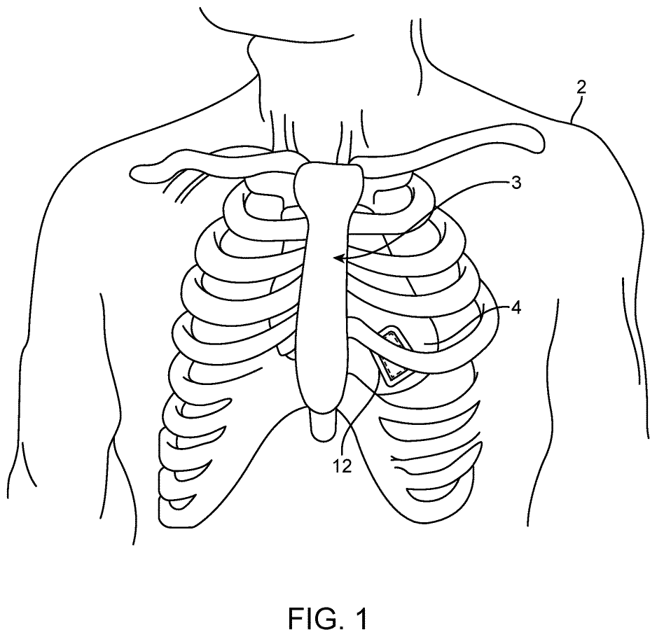

FIG. 1 is a schematic view of one embodiment of an implantable medical device 12 disposed within a body of a patient 2. The device 12 can include any suitable medical device or devices, e.g., a pacing device, pressure sensing device, cardiac monitor, other physiologic sensor, etc. The device 12 can include, e.g., an implantable leadless pacing device that is adapted for implantation entirely within one of the chambers of a heart 4 or on or within a wall of the heart (e.g., epicardium) and that provides electrical signals to the heart beneath a sternum 3 via one or more electrodes carried on the device. The device 12 can also include a surround as is further described herein.

The device 12 is generally described as being attached within a chamber of the heart 4 as an intracardiac pacing device. In one or more embodiments, the device 12 can be attached to an epicardium of the heart 4. In one or more embodiments, device 12 is attached to an external surface of the heart 4, and one or more components of the device such as an electrode can be in contact with an epicardium of the heart. The device 12 is schematically shown in FIG. 1 attached to a wall of a ventricle of the heart 4 via one or more fixation components (e.g., tines, helix, etc.) that penetrate the tissue. These fixation components can secure the device 12 to the cardiac tissue and maintain contact of an electrode (e.g., a cathode or an anode) with the cardiac tissue. The device 12 can be implanted at or proximate to the apex of the heart. In one or more embodiments, a pacing device may be implanted at other ventricular locations, e.g., on the free-wall or septum, an atrial location, or any location on or within the heart 4.

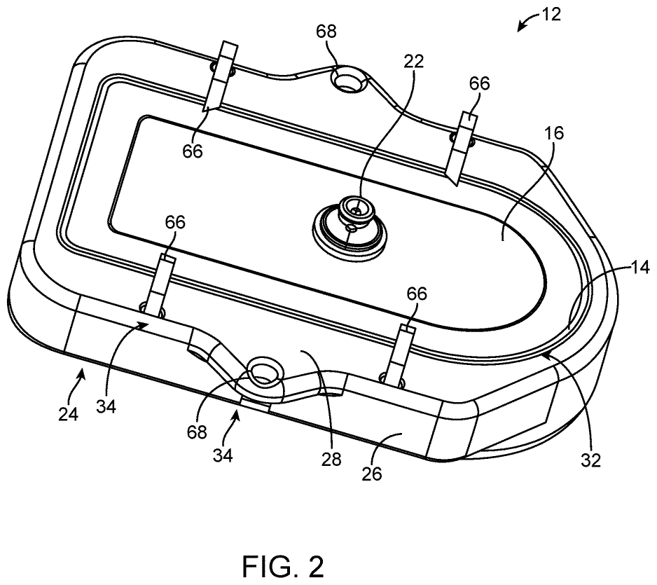

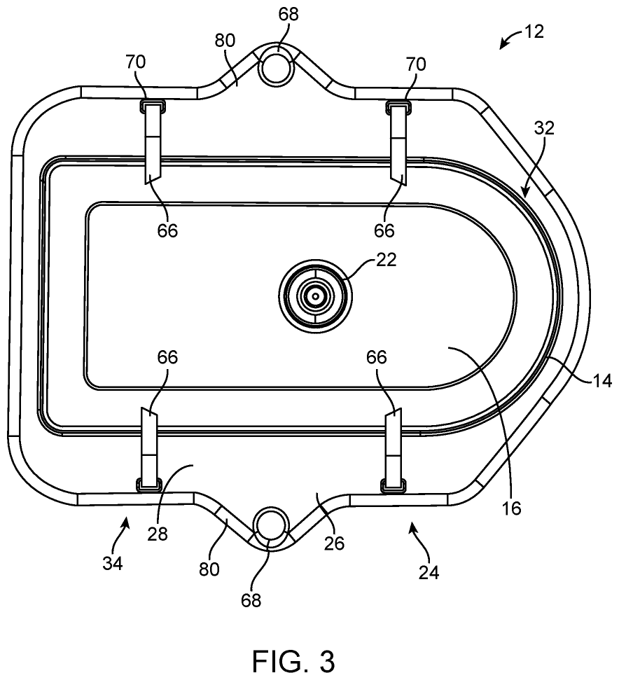

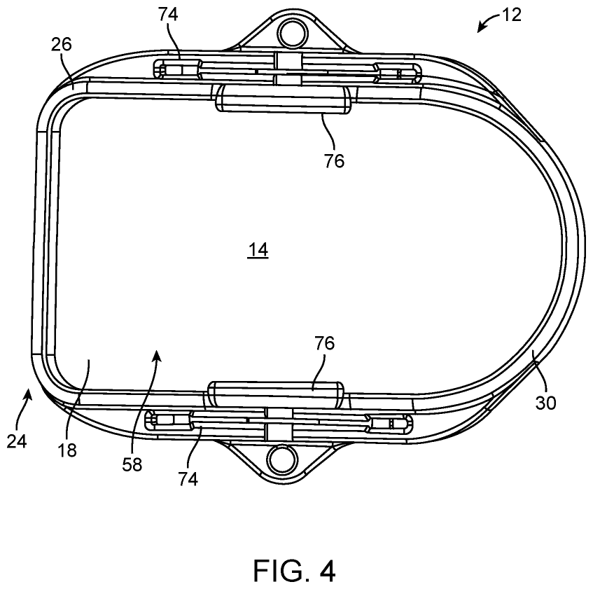

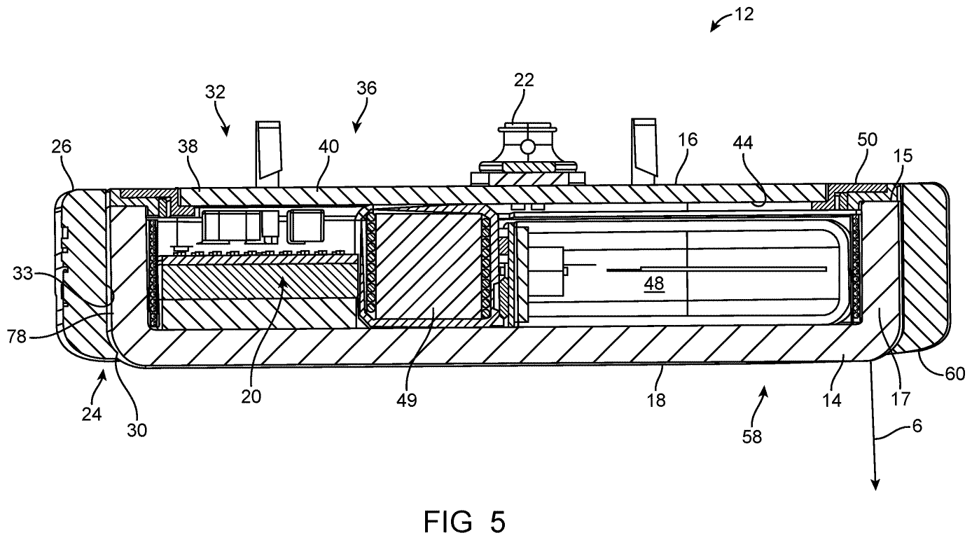

FIGS. 2-5 are various views of the implantable medical device 12 of FIG. 1. The device 12 includes a housing 14 that has a first major surface 16 (FIG. 3) and a second major surface 18 (FIG. 4), electronic components 20 (FIG. 5) disposed within the housing, and an electrode 22 disposed on the first major surface of the housing and electrically connected to the electronic components. The device 12 further includes a surround 24 having a body 26 that includes a first major surface 28 (FIG. 3), a second major surface 30 (FIG. 4), and an opening 32 disposed in the first major surface of the body. The surround 24 is adapted to receive within the body 26 at least a portion of the housing 14 of the IMD 12. The surround 24 further includes one or more fixation components 34 disposed on or through the body 26 of the surround that are adapted to attach the surround to tissue of the patient 2, e.g., the heart 4 (FIG. 1). In one or more embodiments, the electrode 22 of the IMD 12 extends through the opening 32 of the body 26 of the surround 24 and is adapted to be in contact with the tissue of the patient 2.

The device 12 can include any suitable medical device or devices. Representative examples of such IMDs include hearing implants, e.g., cochlear implants; sensing or monitoring devices; signal generators such as cardiac pacemakers or defibrillators, neurostimulators (such as spinal cord stimulators, brain or deep brain stimulators, peripheral nerve stimulators, vagal nerve stimulators, occipital nerve stimulators, subcutaneous stimulators, etc.), gastric stimulators; or the like. In one or more embodiments, the device 12 can include a leadless pacemaker.

The housing 14 of the device 12 can take any suitable shape or shapes and have any suitable dimensions. Further, the housing 14 can include any suitable material or materials, e.g., polymeric materials or inorganic materials such as metallic or ceramic materials. Suitable materials for the housing 14 can include at least one of titanium (e.g., any suitable grade titanium, e.g., grade 1-4 titanium, grade 5 titanium, etc.), stainless steel, polymer, ceramic, glass, or combinations thereof such as laminates, composites, or miscible blends or mixtures. In one or more embodiments, the housing 14 can include any suitable polymeric material or materials, e.g., at least one of epoxy, polyurethane, silicone, polyolefin, acrylic polymer, polyester, polyetheretherketone, polysulfone, polymethylene oxide, or polyvinyl material, or combinations thereof.

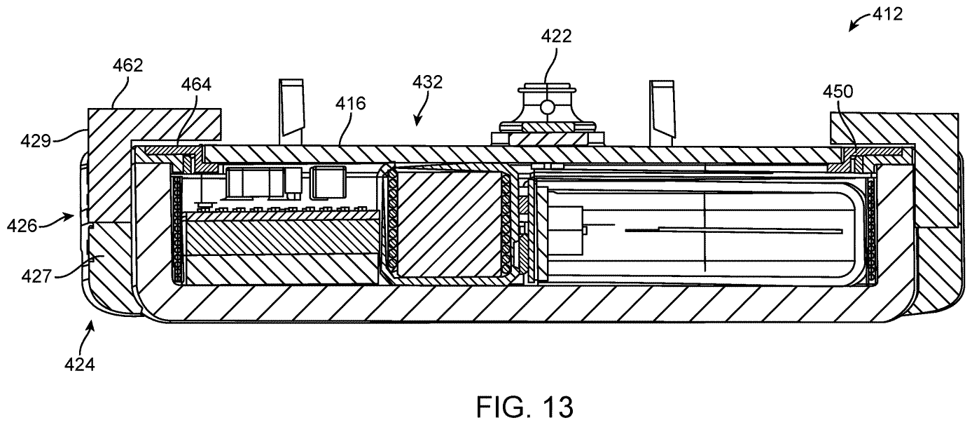

The housing 14 can be a unitary housing. In one or more embodiments, the housing 14 can include two or more portions that are connected using any suitable technique, e.g., welding, mechanically fastening, adhering, thermal bonding, diffusion bonding, laser-assisted diffusion bonding, solvent bonding, etc. See, e.g., surround 424 of FIG. 13. Further, the housing 14 can be formed using any suitable technique, e.g., molding, thermoforming, laminating, over-molding, casting, insert molding, etc.

In one or more embodiments, the device 12 can include a hermetic assembly 36 that forms a portion or part of the housing 14. The hermetic assembly 36 includes a dielectric substrate 38 (FIG. 5) having a first major surface 40 and a second major surface 44, where the first major surface defines at least a portion of the first major surface 16 of the housing 14. Although not shown, the hermetic assembly 36 can also include a patterned layer disposed on at least one of the first major surface 40 or the second major surface 44 of the dielectric substrate 38. The patterned layer can be disposed on the dielectric substrate 38 using any suitable technique, e.g., laser bonding, brazing, diffusion bonding, etc. In one or more embodiments, an interposer (not shown) can be disposed on one or more portions of the patterned layer. The electrode 22 can be disposed on the interposer or patterned layer using any suitable technique.

The hermetic assembly 36 can also include a ferrule 50 that is hermetically sealed to the dielectric substrate 38 using any suitable technique. The ferrule 50 is connected to an edge 15 of a second portion 17 of the housing 14 using any suitable technique. Any suitable hermetic assembly 36 can be utilized with the device 12, e.g., one or more embodiments of hermetic assemblies described in U.S. Patent Publication No. 2021/0178518 to Ruben et al. and entitled HERMETIC ASSEMBLY AND DEVICE INCLUDING SAME. At least a portion of the ferrule 50 can be disposed within the body 26 of the surround 24. In one or more embodiments, the ferrule 50 can be adapted to provide a return path for electrical energy directed to the tissue by the electrode 22.

The dielectric substrate 38 can include any suitable material. In one or more embodiments, the substrate 38 can include a dielectric material, e.g., at least one of glass, quartz, silica, sapphire, silicon carbide, diamond, or gallium nitride. Further, the substrate 38 can include at least one of a biocompatible material or one or more coatings or layers that can provide biocompatibility. The substrate 38 can also take any suitable shape and have any suitable dimensions. The second portion 17 of the housing 14 can also include any suitable material, e.g., the same materials described herein regarding the dielectric substrate 38. The dielectric substrate 38 and the second portion 17 of the housing can include the same materials or different materials.

Further, the ferrule 50 can include any suitable material, e.g., at least one of titanium, niobium, or stainless steel. In one or more embodiments, the ferrule 50 can include a conductive material. The ferrule 50 can take any suitable shape or shapes and have any suitable dimensions.

Disposed within the housing 14 are electronic components 20. The components 20 can include any discrete and/or integrated electronic circuit components that implement analog and/or digital circuits capable of producing the functions attributed to the device 12 described herein. In one or more embodiments, the components 20 can also include components for sensing other physiological parameters, such as acceleration, pressure, sound, and/or impedance. The components 20 can include any suitable electronic component, e.g., at least one of a capacitor, transistor, integrated circuit, including controller or multiplexer, sensor, accelerometer, inductive charging coil, optical components such as emitters and detectors, etc. The device 12 can include any suitable number of electronic components 20. Further, the device 12 can include one or more additional electronic components or elements disposed on an exterior of the housing 14 or on or within the surround 24.

The electronic components 20 can also include a power source (e.g., battery) 48 disposed within the housing 14. The power source 48 can include any suitable power source. In one or more embodiments, the power source 48 is rechargeable battery that can be recharged using any suitable technique using a charging coil 49 that is electrically connected to the battery. Further, the power source 48 can be electrically connected to one or more additional electronic components 20 using any suitable technique.

Disposed on the first major surface 16 of the housing 14 is the electrode 22. Although depicted as being disposed on the first major surface 16, the electrode 22 can be disposed on any suitable portion of the device 12. The electrode 22 can be electrically connected to the electronic components 20 using any suitable technique. The electrode 22 can include any suitable electrode that is adapted to direct electrical energy from the electronic components 20 to tissue of the patient. Further, the electrode 22 can include any suitable electrode that is adapted to receive electrical energy from tissue, e.g., one or more electrical signals from the heart 4. Although depicted as including a single electrode 22, the device 12 can include any suitable number of electrodes disposed on any suitable portions of the device. In one or more embodiments, the electrode 22 extends through the opening 32 of the body 26 of the surround 24 such that the electrode can be in contact with tissue of the patient.

Disposed over one or more portions of the housing 14 of the device 12 is the surround 24, which includes the body 26 having the first major surface 28 and the second major surface 30. The opening 32 of the surround is disposed in the first major surface 28 of the body 26. The surround 24 is adapted to receive within the body 26 at least a portion of the housing 14 of the device 12. The surround 24 can take any suitable shape and have any suitable dimensions.

The body 26 can include any suitable material, e.g., at least one of a polymeric material or inorganic material such as a metallic or ceramic material. In one or more embodiments, the body 26 includes a biocompatible material such as PEEK, PVC, polyethylene, polypropylene, PTFE, PMMA, trimethylcarbonate, medical grade silicone, etc. The body 26 can be a unitary body, e.g., the body can be manufactured as one piece. In one or more embodiments, the body 26 can include two or more portions that are connected together using any suitable technique as is further described herein regarding surround 424 of FIG. 13. Further, the body 26 can be manufactured using any suitable technique, e.g., molding, cast molding, 3D printing, machining, etc.

The opening 32 of the body 26 can be disposed in any suitable portion or portions of the body. As shown, e.g., in FIG. 3, the opening 32 is disposed in the first major surface 28 of the body 26. The opening 32 can take any suitable shape and have any suitable dimensions such that at least a portion of the housing 14 can be received by the surround 24. In one or more embodiments, the opening 32 of the body 26 is adapted to receive the housing 14 such that at least one side surface 78 of the housing is disposed within the surround as shown in FIG. 5. In one or more embodiments, each side surface 78 of the housing 14 can be disposed entirely within the body 26. In one or more embodiments, a portion or portions of one or more of the side surfaces 78 of the housing can be disposed within the body 26.

The opening 32 can include a sidewall 33 (FIG. 5) that extends between the first major surface 28 and the second major surface 30 of the body 26. The sidewall 33 can take any suitable shape. The sidewall 33 is adapted to engage at least a portion of the side surface 78 of the housing 14. In one or more embodiments, the sidewall 33 can include a texture that can aid in retaining the housing 14 at least partially within the surround 24.

In one or more embodiments, the surround 24 also includes a second opening 58 (FIG. 4) that is disposed in the second major surface 30 of the body 26. As can be seen in FIG. 5, a perimeter 60 of the second opening 58 overlaps the housing 14 in a direction 6 orthogonal to the second major surface 30 of the body 26 such that the housing is prevented from being removed from the surround 24 through the second opening. In one or more embodiments, the surround 24 can include one or more tabs 76 (FIG. 4) that extend from the perimeter 60 of the second opening 58 that are adapted to engage the second major surface 18 of the housing 14 to prevent the housing from being directed through the second opening. The tabs 76 can be disposed in any suitable portion or portions of the perimeter 60. Further, the tabs 76 can take any suitable shape and have any suitable dimensions. Although depicted as including two openings 32, 58, the surround 24 can include any suitable number of openings disposed in any suitable portion or portions of the body 26, e.g., one, two, three, four, five, or more openings.

As mentioned herein, the body 26 can take any suitable shape. For example, FIG. 13 is a schematic cross-section view of another embodiment of an implantable medical device 412. All design considerations and possibilities described herein regarding the implantable medical device 12 of FIGS. 1-6 apply equally to the implantable medical device 412 of FIG. 13. One difference between device 412 and device 12 is that device 412 includes a surround 424 having a body 426 that includes a first portion 427 and a second portion 429 connected to the first portion. In one or more embodiments, the first portion 427 of the body 426 can be integral with the second portion 429, i.e., manufactured as a single piece. In one or more embodiments, the first portion 427 can be manufactured separately from the second portion 429 and connected to the second portion using any suitable technique, e.g., press-fitting, mechanically fastening, adhering, welding, bonding, etc. The second portion 429 of the body 426 can be connected to the first portion 427 after a housing 414 of the device 412 has been disposed at least partially within the first portion or the second portion. In one or more embodiments, the first portion 427 can include a material that is different from a material of the second portion 429. In one or more embodiments, the first portion 427 includes the same material as the second portion 429.

The second portion 429 of the body 426 can include a lip 462 that can be adapted to be disposed on a perimeter 464 of a first major surface 416 of a housing 414 of the IMD 412 that faces through an opening 432 of the surround 424 such that at least a portion of the perimeter of the first major surface of the housing is covered by the lip. The lip 462 can extend along any suitable portion of the perimeter 464 of the first major surface 416 of the housing 414. In one or more embodiments, the lip 462 extends over the entire perimeter 464 of the first major surface 416 of the housing 414. The lip 462 can be adapted to retain the housing 414 within the surround 424 once the housing has been disposed at least partially within the surround. In one or more embodiments, the lip 462 can be adapted to reduce a profile of a ferrule 450 of the housing 414 in a plane parallel to the first major surface 416 of the device 412 such that an electrical return path through the ferrule is reduced, thereby adjusting the current flow from an electrode 422 of the device 412 to the ferrule via tissue of the patient.

Returning to FIGS. 1-6, any suitable portion or portions of the housing 14 can be disposed within the surround 24 using any suitable technique. In one or more embodiments, the housing 14 can be press fit into the surround 24 using any suitable technique. Further, the housing 14 can be retained within the surround 24 using any suitable technique. In one or more embodiments, the housing 14 is retained within the surround 24 by friction, i.e., the opening 32 of the body 26 of the surround can have a cross sectional area in a plane parallel to the first major surface 28 of the body that is less than a cross sectional area of the housing 14 in the same plane such that the body retains the housing by friction between the housing and the body. In one or more embodiments, the housing 14 can be adhered to the body 26 using any suitable adhesive. Further, in one or more embodiments, the housing 14 can be mechanically fastened to the body 26 using any suitable technique. In one or more embodiments, the body 26 of the surround 24 can be molded or printed onto the housing 14 using any suitable technique.

The surround 24 further includes the fixation component or components 34 that can be disposed on or through the body 26 of the surround and can be adapted to attach the surround to tissue of the patient. The fixation components 34 can include any suitable component or components that are adapted to connect the surround 24 to tissue. In one or more embodiments, the fixation component 34 includes one or more tines 66. Any suitable number of tines 66 can be disposed on or through the body 26 of the surround 24. In one or more embodiments, the tines 66 can be connected to the body 26 using any suitable technique, e.g., adhered, mechanically fastened, etc. In one or more embodiments, one or more of the tines 66 can be disposed through the body 26. For example, the body 26 can include one or more tine openings 70 disposed through the body 26 such that one or more of the tines 66 can be inserted through the body and retained.

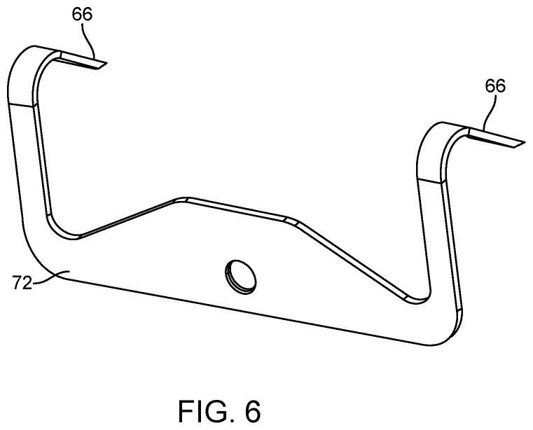

Each tine 66 can take any suitable shape and have any suitable dimensions. In one or more embodiments, each tine 66 can be an individual tine that is connected to the body 26. Further, in one or more embodiments, two or more of the tines 66 can be connected together by a tine body 72 as shown in FIG. 6. The connected tines 66 can be inserted through adjacent tine openings 70, and the tine body 72 can be inserted into a slot 74 disposed in the body 26 of the surround 24 as shown in FIG. 4 such that the tine body is recessed within the surround.

The fixation component 34 can also include one or more suture holes 68 disposed through the body 26 using any suitable technique. The surround 24 can include any suitable number of suture holes 68. As shown in FIGS. 1-6, the surround 24 includes two suture holes 68. In one or more embodiments, the surround 24 includes only suture holes 68 and does not include other fixation components such as tines 66. In one or more embodiments, the surround 24 includes only tines 66 and no suture holes 68. Further, in one or more embodiments, the surround 24 includes one or more tines 66 and one or more suture holes 68. Each suture hole 68 can take any suitable shape and have any suitable dimensions such that a clinician can suture the surround 24 to tissue of the patient using any suitable technique. The suture holes 68 can be disposed in any suitable portion or portions of the body 26 of the surround 24. In one or more embodiments, the body 26 can include one or more tabs 80 that extend from the body 26 as shown in FIG. 3. One or more of the suture holes 68 can be disposed through the tab 80. The tabs 80 can be adapted to offset the suture holes 68 from the body 26 of the surround 24 such that the clinician can thread sutures through the suture holes from either the first major surface 28 or the second major surface 30 of the body.

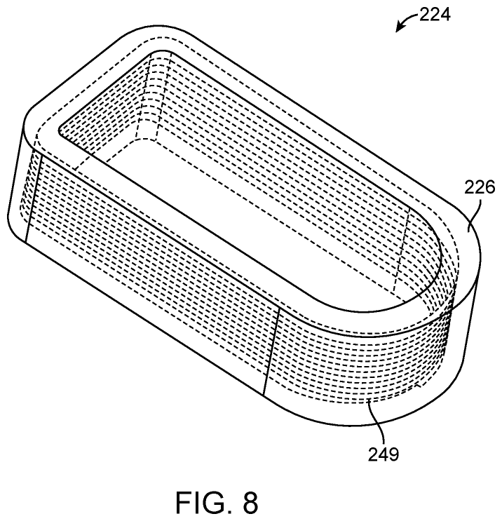

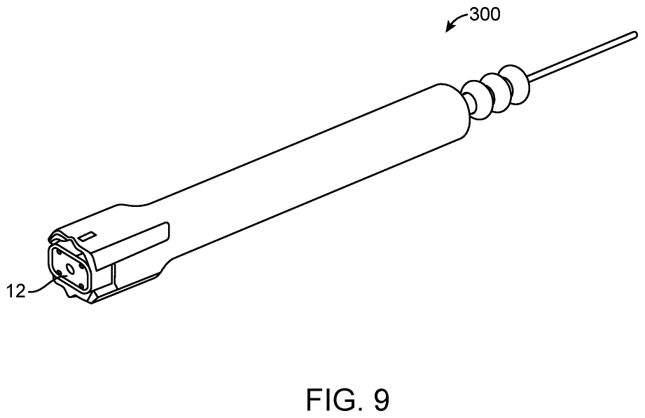

As mentioned herein, one or more electronic components can be disposed on or at least partially withing the surround 24. For example, FIG. 8 is a schematic perspective view of another embodiment of a surround 224. All design considerations and possibilities described herein regarding the surround 24 of FIGS. 1-6 apply equally to the surround 224 of FIG. 8. The surround includes a body 226, which in FIG. 9 is shown as being transparent for descriptive purposes. The surround 224 also includes a charging coil 249 disposed within the body 226. The charging coil 249 can include any suitable charging coil or coils. Further, the charging coil 249 can be disposed within any suitable portion or portions of the body 226 of the surround 224 and electrically connected to one or more electronic components (e.g., electronic components 20 of FIG. 5) disposed on or at least partially within an implantable medical device (e.g., implantable medical device 12 of FIGS. 1-5) using any suitable technique. For example, in one or more embodiments, one or more feedthroughs may be electrically connected to the charging coil 249 that extend from a surface of the body 226 and electrically connect the charging coil to one or more electronic components of an implantable medical device. Although depicted as being disposed within the body 226, one or more portions of the charging coil 249 can be disposed on one or more outer surfaces of the body 226. In one or more embodiments, the charging coil 249 can be completely disposed on one or more outer surfaces of the body 226.

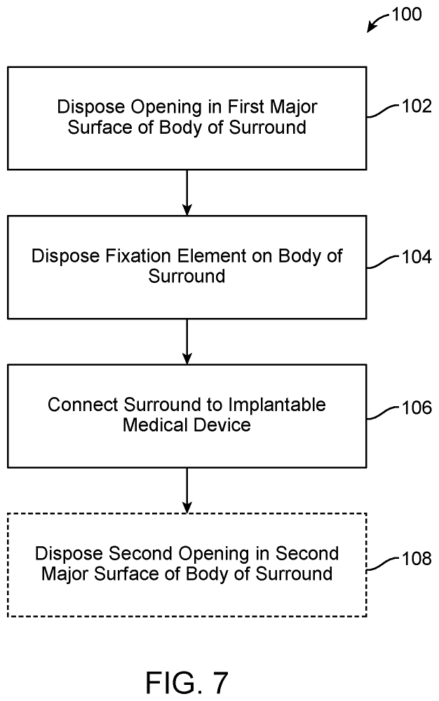

Any suitable technique can be utilized to manufacture the implantable medical device 12. For example, FIG. 7 is a flowchart of one embodiment of a technique 100 for assembling the device 12. Although described regarding the device 12 of FIGS. 1-6, the technique 100 can be utilized to provide any suitable implantable medical device.

At 102, the technique 100 includes disposing the opening 32 in the first major surface 28 of the body 26 of the surround 24 using any suitable technique. In one or more embodiments, the surround 24 is molded such that it includes the opening 32. One or more fixation components 34 can be disposed on or through the body 26 of the surround 24 at 104 using any suitable technique. In one or more embodiments, the fixation component 34 such as the suture holes 68 can be molded into the body 26. Further, in one or more embodiments, the fixation component 34 such as tines 66 can be inserted through the body 26.

At 106, the surround 24 can be connected to the housing 14 of the device 12 using any suitable technique. In one or more embodiments, at least a portion of the housing 14 is disposed within the body 26 of the surround 24. Further, in one or more embodiments, one or more side surfaces 78 of the housing 14 can be disposed within the body 26. The housing 14 is connected to the surround 24 such that the electrode 22 extends through the opening 32 of the body 26 of the surround. At 108, the second opening 58 can optionally be disposed in the second major surface 30 of the body 26 of the surround 24 using any suitable technique such that the perimeter 60 of the second opening overlaps the housing 14 of the device 12 in the direction 6 orthogonal to the second major surface 30 of the body 26. As a result, the device 12 can be prevented from being removed from the surround 24 through the second opening 58 when the housing 14 is disposed at least partially within the surround.

Any suitable technique can be utilized to dispose the device 12 within the body of the patient such that the surround 24 is connected to tissue of the patient. For example, FIGS. 9-12 are various views of one embodiment of an insertion tool 300 that can be utilized to implant the device 12 at a desired location within the body of the patient. Although described regarding the device 12 of FIGS. 1-6, the insertion tool 300 can be utilized to implant any suitable implantable medical device. The insertion tool 300 includes a body 302 having a first end 304 and a second end 306, and a plunger 308 that is disposed at least partially within a channel 310 of the body. The implantable medical device 12 can be disposed at least partially within a cavity 312 of the tool 300. The plunger 308, which extends between a first end 314 and a second end 316, includes a hammer 318 that is connected to the second end of the plunger and is adapted to engage the device 12 and direct the device from the cavity 312 of the body 302 of the tool 300.

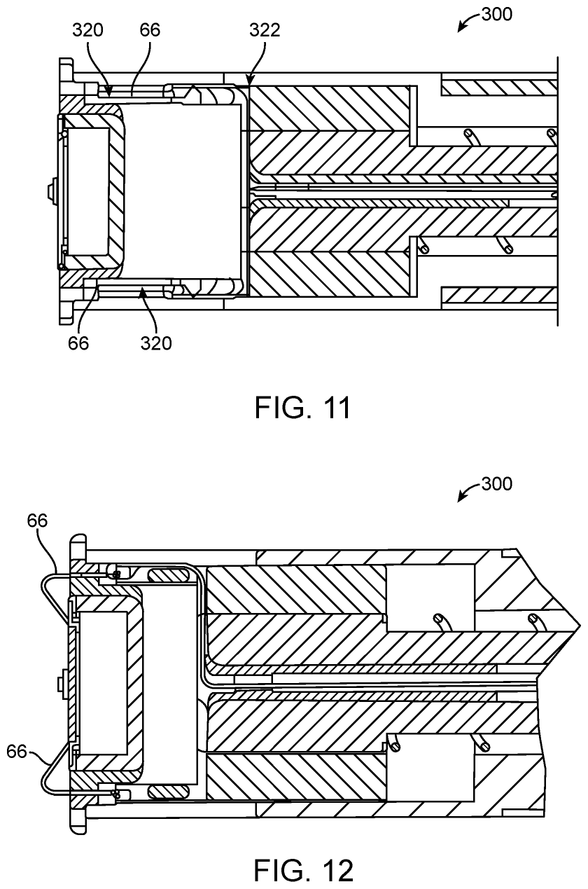

As can be seen in FIGS. 11-12, the body 302 also includes tine channels 320 that are adapted to receive the tines 66 of the surround 24 of the IMD 12. The tine channels 320 can be aligned with the slots 74 and tine openings 70 disposed in the body 26 of the surround such that the tines 66 can be directed from the tine channels 320 into the slots and through the openings.

The tool 300 can also include one or more retention sutures 322 that can be connected to the plunger 308. The retention sutures 322 can also be connected to the tines 66 such that when the device 12 is disposed within the cavity 312 and the plunger 308 is pulled in a direction away from the first end 304 of the body 302 of the tool 300, the tines 66 are retracted into the tine channels 320. When retracted into these channels 320, the tines 66 are straightened as can be seen in FIG. 11. The straightened tines 66 are, therefore, prepared for deployment into tissue of the patient. In one or more embodiments, the tines 66 can be biased in a bent configuration for securing the IMD 12 to tissue. Because of this bias, the tines 66 may need to be straightened as shown in FIG. 11 for deployment.

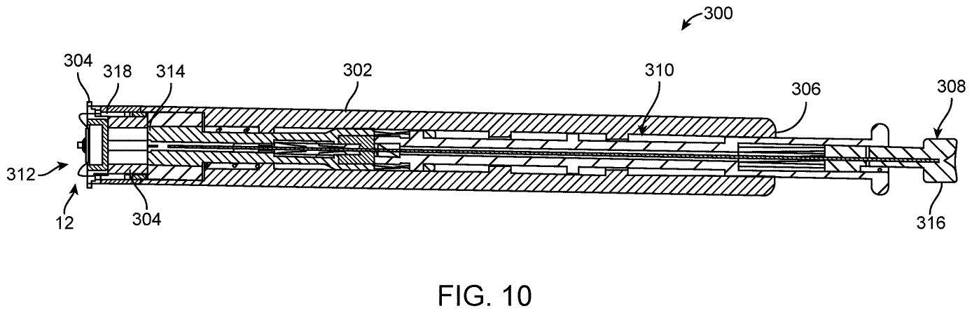

FIGS. 10 and 12 show the tines 66 after they have been deployed into tissue (not shown). As such, the tines 66 return to their biased curved configuration to retain the device 12 on the tissue. The tines 66 and the device 12 can be deployed using any suitable technique. In one or more embodiments, the plunger 308 is directed toward the first end 304 of the body 302 of the tool 300 such that the hammer 318 forces the device 12 from the cavity 312 and into contact with the tissue of the patient. The retention sutures 322 are released using any suitable technique such that the clips can extend beyond the channels 320 into the slots 74 and tine openings 70 of the surround 24 where they can extend through the openings of the surround.

To utilize the insertion tool 300 for deployment of the device 12, the tines 66 can be inserted into the slots 74 of the body 26 of the surround. The retention sutures 322 can be connected to the tines 66 using any suitable technique. The IMD 12 can then be disposed within the cavity 312 and the tines 66 retracted into the tine channels 320 such that they are in a straight configuration as shown in FIG. 11. The insertion tool 300 can be directed to an implantation site within the patient (e.g., the epicardium) using any suitable technique. When the first end 304 of the body 302 of the tool 300 is disposed adjacent to the implantation site and the device 12 is in contact with the tissue at the site, the plunger 308 can be directed toward the first end 304 of the body 302 such that the tines 66 are directed through the channels 320 and the slots 74 of the surround 24 and into engagement with the target tissue. Once the tines 66 extend from the tine openings 70 of the surround 24, they take on their biased curved shape as shown in FIGS. 10 and 12 such that the tines secure the device 12 to the target tissue. The hammer 318 of the plunger 308 is adapted to direct the device 12 against the target tissue and beyond the cavity 312. The retention sutures 322 can be released from the tines 66 using any suitable technique such that the insertion tool 300 is no longer connected to the device 12. The tool 300 can be removed from the patient using any suitable technique.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

1. An implantable medical device comprising:
a housing comprising a first major surface and a second major surface;
electronic components disposed within the housing;
an electrode disposed on the first major surface of the housing and electrically connected to the electronic components; and
a surround comprising a body that comprises a first major surface, a second major surface, and an opening disposed in the first major surface of the body, wherein the surround is adapted to receive within the body at least a portion of the housing, wherein the surround further comprises a fixation component disposed on or through the body of the surround and adapted to attach the surround to tissue of a patient;
wherein the electrode extends through the opening of the body of the surround and is adapted to be in contact with the tissue of the patient.

2. The device of claim 1, wherein the fixation component comprises a tine.

3. The device of claim 1, wherein the fixation component comprises a suture hole.

4. The device of claim 1, wherein the body of the surround comprises a biocompatible material.

5. The device of claim 4, wherein the biocompatible material of the body of the surround comprises PEEK.

6. The device of claim 1, wherein the opening of the body of the surround is adapted to receive the housing such that at least one side surface of the housing is disposed within the surround.

7. The device of claim 6, wherein each side surface of the housing is disposed within the body of the surround.

8. The device of claim 1, wherein the surround further comprises a second opening disposed in the second major surface of the body of the surround, wherein a perimeter of the second opening overlaps the housing in a direction orthogonal to the second major surface of the body such that the housing is prevented from being removed from the surround through the second opening.

9. The device of claim 1, wherein the body of the surround further comprises a lip disposed on the first major surface of the body of the surround that defines the opening, wherein the lip is adapted to be disposed on a perimeter of the first major surface of the housing that faces through the opening such that at least a portion of the perimeter of the first major surface of the housing is covered by the lip.

10. The device of claim 1, wherein the surround is adapted such that the housing is press-fit into the body of the surround.

11. The device of claim 1, wherein the housing is adhered to the body of the surround.

12. The device of claim 1, wherein the body of the surround comprises a first portion and a second portion that is connected to the first portion.

13. The device of claim 1, further comprising a hermetic assembly that forms a part of the housing, wherein the hermetic assembly comprises:
a dielectric substrate comprising a first major surface and a second major surface, wherein the first major surface of the dielectric substrate defines at least a portion of the first major surface of the housing; and
a ferrule hermetically sealed to the dielectric substrate;
wherein the ferrule is connected to an edge of a second portion of the housing.

14. The device of claim 13, wherein the ferrule is laser bonded to the dielectric substrate.

15. The device of claim 13, wherein at least a portion of the ferrule is disposed within the body of the surround.
